(19) United States
(12) Patent Application Publication
(10) Pub. No.: US 2007/0150027 A1
(43) Pub. Date: Jun. 28, 2007

(54) Non-invasive device and method for electrical stimulation of neural tissue

(76) Inventor: Lesco L. Rogers, Raleigh, NC (US)

Correspondence Address: Miller, Canfield, Paddock and Stone P.L.C.;c/o Robert Kelley Roth, Suite 2500, 150 West Jefferson Ave., Detroit, MI, 48226, US

(21) Appl. No.: 11/315,918

(22) Filed: Dec. 22, 2005

(51) Int. Cl. A61N 1/00

(52) U.S. Cl. 607/55

(57) ABSTRACT

A non-invasive device and method for stimulation of neural tissue of a patient comprises a power source and at least one pair of electrodes. The power source applies a voltage to the pair of electrodes, and the electrodes extend through a top layer of skin generally adjacent Arnold's nerve. The device can be formed in shape similar to a hearing aid. A second pair of electrodes may also be used, positioned on the skin generally adjacent the auriculotemporal nerve.

### FIELD OF THE INVENTION

This invention relates to improvements in devices and methods for stimulating neural tissue, and more particularly to non-invasive devices and methods for electrically stimulating neural tissue.

### BACKGROUND OF THE INVENTION

Electrical stimulation of the vagus nerve has been shown for some patients to enhance brain function. Enhanced brain function can comprise, for example, improved memory, better learning consolidation processes, quicker recovery of function following injury, enhanced cognitive processing, motor and perceptual skills, treating persistent impairment of consciousness, and preventing epilepsy and symptoms associated with the disease, etc. The exact mechanism is not completely understood, but is believed to involve transmission of electrical stimulation of the vagus nerve to various regions of the brain, including the cingulated gyrus, thalamus, hippocampus, and amygdala, etc. U.S. Pat. No. 6,556,868 to Naritoku et al, the disclosure of which is expressly incorporated herein in its entirety by reference, discusses some of the research in this area and discloses an implantable device for stimulating the vagus nerve. This direct stimulation requires surgery to install the necessary electrodes. Cyberonics, Inc. is a Texas corporation which has also developed an implantable device for VNS (Vagus Nerve Stimulation) Therapy for the treatment of epilepsy or chronic and recurrent depression. An electrode formed as a wire is wound around the vagus nerve (10^{th} Cranial nerve) in the neck.

Implantable devices has obvious limitations, including trauma to the body and cost. Moreover, implantable devices for neural stimulation may be limited in the amount and duration of voltage which can be applied, which can limit the therapeutic effect of such devices. These problems include voice alteration, increased cough, shortness of breath, neck pain, difficulty swallowing, nausea, and inflammation of the throat. Also, patients with implanted devices must avoid electrical or electromechanical devices with a strong magnetic field, as such fields can potentially trigger a battery to start or may cause a change in settings. Also long term implantation of leads on the vagus nerve has been associated with scarring.

A paper by Fallgatter et al in J Nerual Transm (2003) 110:1437-1443, discusses stimulation of the vagus nerve using electrodes pasted on to the skin. However, such electrodes are uncomfortable, and the skin may be irritated or burned when voltages are applied for a time and amplitude sufficient to produce a therapeutic effect. It would be desirable to provide an improved device for stimulation of neural tissue which is of low cost, minimally invasive and which provides a therapeutic effect.

### SUMMARY OF THE INVENTION

In accordance with a first aspect, a non-invasive device for stimulation of neural tissue of a patient comprises a power source and at least one pair of electrodes. The power source applies a voltage to the pair of electrodes, and the electrodes extend through a top layer of skin generally adjacent Arnold's nerve. The device can be formed in shape similar to a hearing aid.

In accordance with another aspect, a second pair of electrodes may also be used, positioned on the skin generally adjacent the auriculotemporal nerve. In accordance with another aspect, a method of stimulating neural tissue comprises positioning electrodes generally adjacent an Arnold's nerve of a patient, wherein the electrodes extend at least through a stratum corneum layer of an epidermis of the patient, and applying a voltage across the electrodes.

From the foregoing disclosure and the following more detailed description of various preferred embodiments it will be apparent to those skilled in the art that the present invention provides a significant advance in the technology of brain function enhancement. Particularly significant in this regard is the potential the invention affords for providing a high quality, low cost, non-invasive device and method for enhancement of brain function. Additional features and advantages of various preferred embodiments will be better understood in view of the detailed description provided below.

### BRIEF DESCRIPTION OF THE DRAWINGS

FIG. 1 is a side view of a device for stimulation of neural tissue in accordance with a preferred embodiment, positioned in a left ear of a person.

FIG. 2 is a view of an electrode in accordance with a preferred embodiment where each electrode is provided with a series of projections.

FIG. 3 is a schematic showing electrical stimulation of neural tissue in accordance with a preferred embodiment, initiated at the ear and indirectly stimulating various portions of the nervous system and brain.

FIG. 4 shows various portions of the brain which are stimulated by the device.

FIG. 5 shows an alternate preferred embodiment wherein multiple pairs of electrodes are used for neural stimulation at multiple locations.

It should be understood that the appended drawings are not necessarily to scale, presenting a somewhat simplified representation of various preferred features illustrative of the basic principles of the invention. The specific design features of the device for stimulating neural tissue as disclosed here, including, for example, the specific dimensions of the electrodes, will be determined in part by the particular intended application and use environment. Certain features of the illustrated embodiments have been enlarged or distorted relative to others to help visualization and clear understanding. In particular, thin features may be thickened, for example, for clarity of illustration. All references to direction and position, unless otherwise indicated, refer to the orientation illustrated in the drawings.

### DETAILED DESCRIPTION OF CERTAIN PREFERRED EMBODIMENTS

It will be apparent to those skilled in the art, that is, to those who have knowledge or experience in this area of technology, that many uses and design variations are possible for the device for stimulating neural tissue disclosed herein. The following detailed discussion of various alternative and preferred features and embodiments will illustrate the general principles of the invention with reference to a non-invasive device for stimulating neural tissue mounted in a manner similar to an ear piece. Other embodiments suitable for other applications will be apparent to those skilled in the art given the benefit of this disclosure.

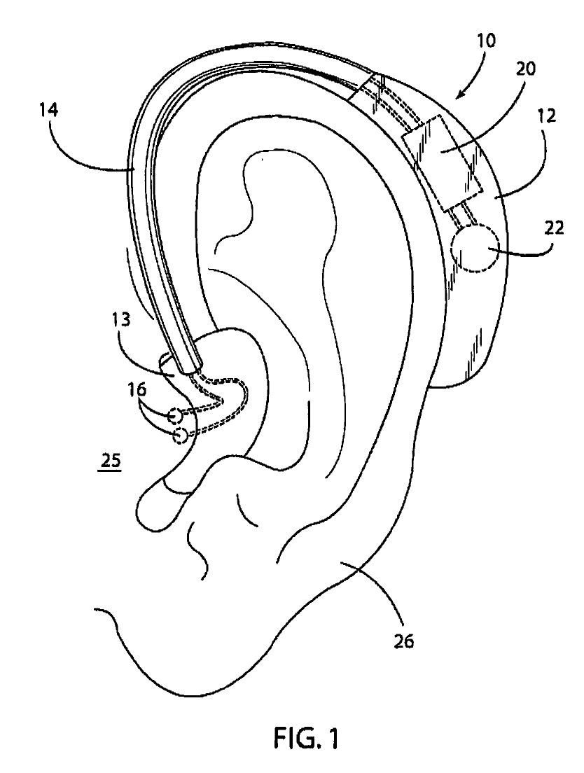

Referring now to the drawings, in FIG. 1 shows a neural stimulation device 10 in accordance with a preferred embodiment. A pair of electrodes 16 is positioned in a housing 13, positioned in an ear of a patient. The housing may preferably be shaped much like an earpiece. The electrodes 16 are electrically connected via connector 14 to a behind the ear case 12 containing a power source 22, typically a battery, and a controller/timing circuit 20 which regulates the amplitude, duration, interval and frequency of an electrical pulse supplied to the electrodes. Preferably the device 10 is placed in the left ear of the patient, as the nerves that are stimulated in the left ear have relatively fewer connections to other organs and tissues besides the brain. In particular, the left vagus nerve has fewer connections to the heart, and therefore effects the heart to a lesser degree than the right vagus nerve.

Preferably the housing 13 extends into the auditory canal of the ear of the patient. Positioned as shown in FIG. 1, the electrodes 16 extend out of the housing 13 and into an area of skin behind the tragus 25, generally adjacent the Arnold's nerve of the patient.

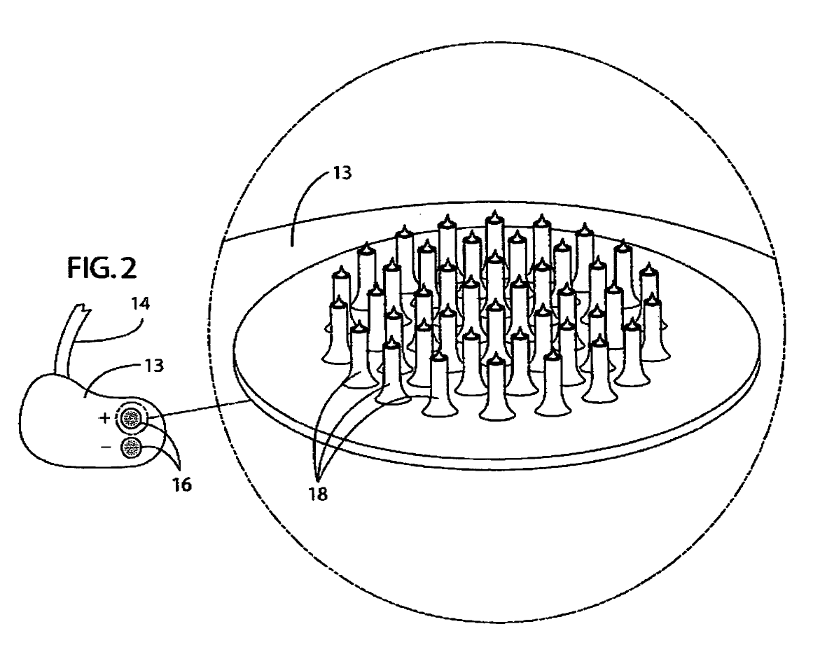

A preferred electrode would be, for example a dry, spiked electrode such as those provided by Orbital Research Inc. of Cleveland, Ohio. Such dry electrodes are highly advantageous over known pre-gelled based electrodes in that patients tend to develop skin irritations with application of gel electrodes and that gel electrodes dry out. Preferably the electrodes are non-invasive in the sense that surgery or injections are not required, and the electrodes do not extend into the layers beneath the epidermis layer of the skin which may contain nerve fibers. Rather, preferably the electrodes penetrate at least and through the stratum corneum of the epidermis layer of the skin. As shown in FIG. 2, the electrodes may be provided with a series of spikes or projections 18 which penetrate a top layer of the skin (at least the stratum corneum), advantageously reducing the amount of resistance and extending the battery life. Most preferably the projections 18 of the electrode extend down to the stratum germinativum or just above the top layer of the dermis, above the layers containing nerve fibers. Such a design is also advantageous in that the patient should have reduced or preferably minimal sensation when the voltage is applied across the electrodes.

Preferably the power source is a battery such as the Auria™ PowerCel™ Plus rechargeable battery made by Advanced Bionics, a Boston Scientific Company. The controller or timing circuit can regulate various parameters of the voltage applied across the electrodes, including, for example, the current, amplitude, pulse width, frequency, and duration of pulses. A therapeutic effect may be achieved with an amplitude of the voltage applied at the electrodes 16 in the range of 0.5-10 Volts, the pulse width in the range of 90-500 μsec, the frequency in the range of 15-150 Hz. In accordance with a highly advantageous feature, the duration of application of the voltage can be extended for much longer than with known devices. Because the patient can tolerate this device so well, the duration of application of voltage may be extended as needed to enhance brain function for a very wide range of patients.

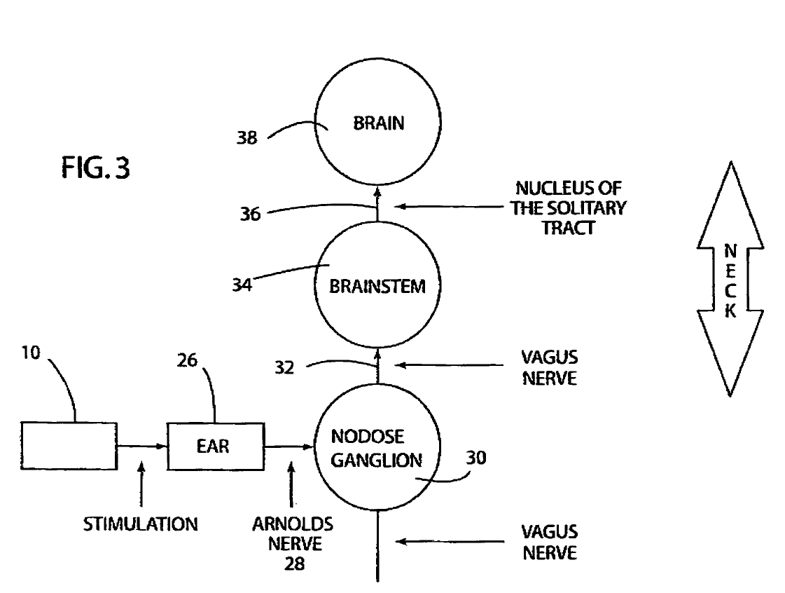

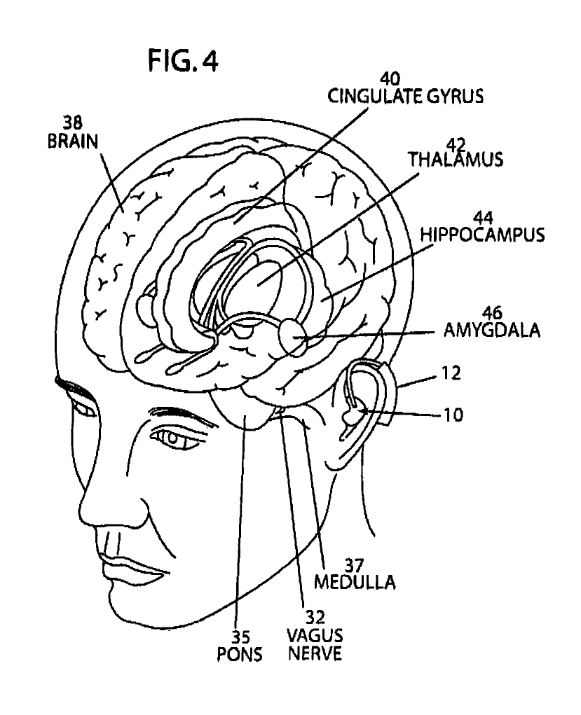

FIGS. 3-4 show a general schematic showing the flow of electrical stimulation to the brain. Instead of directly stimulating the vagus nerve, electrical stimulation is provided in the auditory canal near the ear 26 (most preferably generally adjacent Arnold's nerve 28), and from there is transmitted to the nodose ganglion 30, to the vagus nerve (Cranial Nerve X) 32, to the brainstem 34, to the solitary nucleus 36, and to the brain 38. FIG. 4 shows the various portions of the brain 38 which would be stimulated by application of the voltage by device 10, including the cingulated gyrus 40, thalamus 42, hippocampus 44 and amygdala 46.

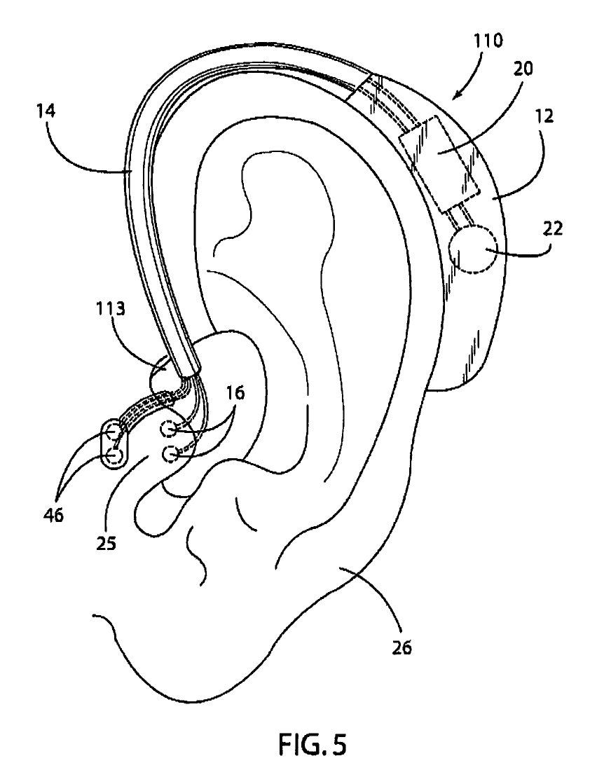

FIG. 5 shows an alternate preferred embodiment of a neural stimulation device 110 where a second pair of electrodes 46 are positioned in a modified housing 13. The first pair of electrodes 16 is positioned generally as before, preferably near the Arnold's nerve of the patient. The second pair of electrodes, preferably similar in composition to the first pair of electrodes, extend out of the ear 26 and are positioned on the side of the cheek, generally adjacent to the location of the auriculotemporal nerve, as shown in FIG. 5. Electrical stimulation of the auriculotemporal nerve may be accomplished in a manner similar to electrical stimulation of Arnold's nerve. Stimulation of the auriculotemporal nerve would in turn stimulate the trigeminal nerve (Cranial Nerve V) and from there stimulate the various parts of the brain. Voltages applied and duration may be applied as before, and may also be applied independently of the voltage applied generally adjacent Arnold's nerve. Optionally additional electrodes may be used, if necessary. For example, it will be readily apparent to those skilled in the art, given the benefit of this disclosure, that electrodes may be positioned generally adjacent the glossopharyngeal nerve, also near the ear, for electrical stimulation of this nerve.

It will be readily apparent to those skilled in the art, given the benefit of this disclosure, that not only may the neural tissue stimulating device be used for providing a therapeutic effect for treatment of diseases such as epilepsy, Alzheimers, Parkinsons, migraine headaches, anxiety disorders, brain injuries and related trauma, depression (including postpartum depression) etc., but also for enhancement of normal patients. For example, use of this device with normal patients may lead to heightened states of awareness and enhanced memory function. Because the neural tissue stimulation device is non-invasive and relatively low cost, the device may advantageously dramatically expand the field of ‘cosmetic neurology’ where normal patients use the device to help them learn new areas of study quicker and better, for example.

From the foregoing disclosure and detailed description of certain preferred embodiments, it will be apparent that various modifications, additions and other alternative embodiments are possible without departing from the true scope and spirit of the invention. The embodiments discussed were chosen and described to provide the best illustration of the principles of the invention and its practical application to thereby enable one of ordinary skill in the art to use the invention in various embodiments and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. A non-invasive device for stimulation of neural tissue of a patient comprising, in combination:
a power source; and
at least one pair of electrodes;
wherein the power source applies a voltage to the at least one pair of electrodes, and the electrodes extend through a top layer of skin generally adjacent the auditory canal of the patient.

2. The non-invasive device of claim 1 wherein the electrodes extend through a stratum corneum layer of an epidermis layer of the skin.

3. The non-invasive device of claim 2 wherein the electrodes extend to a stratum germinativum layer of the skin.

4. The non-invasive device of claim 1 further comprising another pair of electrodes generally positioned generally adjacent the auriculotemporal nerve of the patient, wherein the electrodes are powered by the power source.

5. The non-invasive device of claim 1 further comprising a controller and a power source mounted in a housing adapted to be positioned behind an ear of the patient.

6. The non-invasive device of claim 5 wherein the pair of electrodes are positioned in a casing and connected to the housing by a connector.

7. The non-invasive device of claim 1 wherein the voltage applied at the at least one pair of electrodes has an amplitude ranging from about 0.5 to 10 Volts.

8. The non-invasive device of claim 1 wherein the voltage applied at the at least one pair of electrodes has a pulse width ranging from about 90 to 500 microseconds.

9. The non-invasive device of claim 1 wherein the voltage applied at the at least one pair of electrodes has a frequency ranging from about 15 to 150 Hz.

10. The non-invasive device of claim 1 wherein the electrodes are provided with a plurality of spikes which extend into the skin.

11. The non-invasive device of claim 1 wherein the at least one pair of electrodes is positioned generally adjacent Arnold's nerve.

12. The non-invasive device of claim 1 wherein the at least one pair of electrodes are positioned near the left ear of the patient.

13. A non-invasive device for stimulation of neural tissue of a patient comprising, in combination:
a power source; and
at least one pair of electrodes;
wherein the power source applies a voltage to the at least one pair of electrodes, and the electrodes extend through a top layer of skin generally adjacent an Arnold's nerve of the patient.

14. The non-invasive device of claim 13 further comprising a second pair of electrodes which extend through a top layer of skin generally adjacent an auriculotemporal nerve of the patient.

15. A non-invasive method of stimulating neural tissue in a patient comprising, in combination, the steps of:
positioning at least one pair of electrodes generally adjacent an Arnold's nerve of a patient, wherein the electrodes extend at least through a stratum corneum layer of an epidermis of the patient; and
applying a voltage across the at least one pair of electrodes.

16. The non-invasive method of stimulating neural tissue of claim 15 wherein the electrodes are formed with a plurality of spikes which extend into the epidermis of the patient.

17. The non-invasive method of stimulating neural tissue of claim 15 wherein the electrodes extend to a stratum germinativum layer of the patient.

18. The non-invasive method of stimulating neural tissue of claim 15 further comprising the step of positioning the electrodes generally adjacent the left ear.
